(19) United States
(12) Patent Application Publication
(10) Pub. No.: US 2010/0010303 A1
(43) Pub. Date: Jan. 14, 2010

(54) INFLATABLE ACCESS DEVICE

(75) Inventor: Gregory J. Bakos, Mason, OH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/169,868

(22) Filed: Jul. 9, 2008

(51) Int. Cl. A61B 1/32

(52) U.S. Cl. 600/115

(57) ABSTRACT

A surgical instrument can be used to dilate a tissue within the body of a patient to create a working space within the body to allow a surgeon to easily advance an endoscope into a patient's body. In various embodiments, the surgical instrument can include a balloon capable of transitioning from a collapsed position to an expanded position and a sleeve at least partially surrounding the balloon when the balloon is in the collapsed position. The sleeve includes at least one frangible portion that may at least partially release along the frangible portion when the balloon transitions from the collapsed position to the expanded position. In various embodiments, the surgical instrument further includes a catheter, a needle, an endoscope or an endoscopic trocar.

### BACKGROUND

i. Field of the Invention

The present invention generally relates to surgical devices and, more particularly, to surgical devices for manipulating tissue within a patient's body.

ii. Description of the Related Art

Traditional, or open, surgical techniques may require a surgeon to make large incisions in a patient's body in order to access a tissue treatment region, or surgical site. In some instances, these large incisions may prolong the recovery time of and/or increase the scarring to the patient. As a result, minimally invasive surgical techniques are becoming more preferred among surgeons and patients owing to the reduced size of the incisions required for various procedures. In some circumstances, minimally invasive surgical techniques may reduce the possibility that the patient will suffer undesirable post-surgical conditions, such as scarring and/or infections, for example. Further, such minimally invasive techniques can allow the patient to recover more rapidly as compared to traditional surgical procedures.

Endoscopy is one minimally invasive surgical technique which allows a surgeon to view and evaluate a surgical site by inserting at least one cannula, or trocar, into the patient's body through a natural opening in the body and/or through a relatively small incision. In use, an endoscope can be inserted into, or through, the trocar so that the surgeon can observe the surgical site. In various embodiments, the endoscope may include a flexible or rigid shaft, a camera and/or other suitable optical device, and a handle portion. In at least one embodiment, the optical device can be located on a first, or distal, end of the shaft and the handle portion can be located on a second, or proximal, end of the shaft. In various embodiments, the endoscope may also be configured to assist a surgeon in taking biopsies, retrieving foreign objects, and introducing surgical instruments into the surgical site.

Laparoscopic surgery is another minimally invasive surgical technique where procedures in the abdominal or pelvic cavities can be performed through small incisions in the patient's body. A key element of laparoscopic surgery is the use of a laparoscope which typically includes a telescopic lens system that can be connected to a video camera. In various embodiments, a laparoscope can further include a fiber optic system connected to a halogen or xenon light source, for example, in order to illuminate the surgical site. In various laparoscopic, and/or endoscopic, surgical procedures, a body cavity of a patient, such as the abdominal cavity, for example, can be insufflated with carbon dioxide gas, for example, in order to create a temporary working space for the surgeon. In such procedures, a cavity wall can be elevated above the organs within the cavity by the carbon dioxide gas. Carbon dioxide gas is usually used for insufflation because it can be easily absorbed and removed by the body.

In at least one minimally invasive surgical procedure, an endoscope and/or laparoscope can be inserted through a natural opening of a patient to allow a surgeon to access a surgical site. Such procedures are generally referred to as Nature Orifice Transluminal Endoscopic Surgery or (NOTES)™ and can be utilized to treat tissue while reducing the number of incisions, and external scars, to a patient's body. In various NOTES procedures, for example, an endoscope can include at least one working channel defined therein which can be used to allow the surgeon to insert a surgical instrument therethrough in order to access the surgical site.

### SUMMARY

According to at least one aspect of the invention, surgical instruments including a frangible sleeve can be utilized to facilitate the entry of a deflated balloon into a surgical site. In various embodiments, as described herein, the frangible sleeve can allow a surgeon to easily introduce a deflated balloon into a surgical site.

In at least one aspect, the invention relates to a surgical instrument for use in manipulating tissue within the body of a patient. In various embodiments, the surgical instrument can include a balloon, and a sleeve, wherein the balloon can be configured to be transitioned, for example, from a collapsed position to an expanded position, wherein the sleeve can include at least one frangible portion, for example, and wherein the sleeve can be configured to at least partially surround the balloon when the balloon is in a collapsed position and to at least partially separate along the at least one frangible portion when the balloon is in an expanded position, for example.

In various embodiments, the at least one frangible portion on the sleeve of the surgical instrument can be configured to extend in various configurations relative to a longitudinal axis of the sleeve. In at least one embodiment, the frangible portion can be configured to extend in at least one line which is parallel, or at least substantially parallel, to the longitudinal axis of the sleeve. In a further embodiment, the frangible portion can be configured to extend in two, three, four or more lines, wherein each line is parallel, or at least substantially parallel, to the longitudinal axis of the sleeve. In another embodiment, the frangible portion can be configured to extend in at least one substantially helical pattern relative to the longitudinal axis of the sleeve. In a further embodiment, the frangible portion can be configured to extend along at least two congruent, or at least substantially congruent, helical patterns relative to the longitudinal axis of the sleeve. In a further embodiment, the at least two congruent, or at least substantially congruent, helical patterns do not intersect along a central portion of the sleeve, for example. In another embodiment, the frangible portion on the sleeve of the surgical instrument can be configured to extend in a pattern, wherein the pattern is substantially parallel to the longitudinal axis of the sleeve along a first portion of the sleeve and substantially helical relative to the longitudinal axis of the sleeve along a second portion of the sleeve. In at least one embodiment, the frangible portion comprises a perforation. In a further embodiment, the frangible portion comprises a score.

In at least one form of the invention, the surgical instrument can further include a catheter configured to be attached to a proximal portion of the balloon, for example, and optionally further configured to be attached to a distal portion of the balloon, for example. In various embodiments, the sleeve can further include an anchor portion at at least one end of the sleeve. In a further embodiment, the anchor portion of the sleeve can be located at a proximal portion of the sleeve and can be configured to be attached to a portion of the catheter adjacent to a proximal portion of the balloon. In an additional embodiment, the sleeve can further include a tapered transition portion at a distal portion of the sleeve configured to be located adjacent to a distal portion of the balloon. In another further embodiment, the anchor portion of the sleeve can be located at a distal portion of the sleeve and can be configured to be attached to a portion of the catheter adjacent to a distal portion of the balloon.

In at least one form of the invention, the surgical instrument can further include an endoscope, for example, including an endoscope lumen extending along a longitudinal axis of the endoscope. According to this embodiment, the balloon and the sleeve can be slidably received within the endoscope lumen. In a further form of the invention, the surgical instrument can further include a trocar, for example, including a trocar lumen extending along the longitudinal axis of the trocar. According to this embodiment, the balloon and the sleeve, and optionally an endoscope, can be slidably received within the trocar lumen.

In another aspect, the invention relates to a surgical kit for use in manipulating tissue within the body of a patient. In at least one form of the invention, the kit can include a balloon, a sleeve, a catheter, and an endoscope, wherein the balloon can be configured to be transitioned from a collapsed position to an expanded position, wherein the sleeve can include at least one frangible portion and wherein the sleeve can be configured to at least partially surround the balloon when the balloon is in the collapsed position and to at least partially release along the at least one frangible portion when the balloon is in the expanded position, wherein the catheter can be configured to be attached to at least a portion of the balloon, and wherein the endoscope further includes an endoscope lumen and wherein the catheter, balloon and sleeve can be slidably received within the endoscope lumen. In a further embodiment, the surgical kit can further include a trocar, wherein the trocar can include a trocar lumen extending along the longitudinal axis of the trocar and wherein the endoscope can be slidably received within the trocar lumen.

### BRIEF DESCRIPTION OF THE FIGURES

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a diagram of a surgical instrument including a catheter, a balloon, a frangible sleeve and a needle in accordance with one non-limiting embodiment of the present invention;

FIG. 2 is a diagram of a sleeve including linear frangible portions in accordance with one non-limiting embodiment of the present invention;

FIG. 3 is a diagram of a sleeve including a helical frangible portion in accordance with one non-limiting embodiment of the present invention;

FIG. 4 is a diagram of a sleeve including two congruent helical frangible portions in accordance with one non-limiting embodiment of the present invention;

FIG. 5 is a diagram of a sleeve including frangible portions that are linearly arranged along a first portion and helically arranged along a second portion in accordance with one non-limiting embodiment of the present invention;

FIG. 6 is a diagram of a surgical instrument including an endoscopic trocar, an endoscope, a catheter, a balloon, a sleeve and a needle after the needle has been inserted through a tissue wall in accordance with one non-limiting embodiment of the present invention;

FIG. 7 is a diagram of the surgical instrument of FIG. 6 after the balloon and sleeve have been inserted into the tissue wall in accordance with one non-limiting embodiment of the present invention;

FIG. 8 is a diagram of the surgical instrument of FIG. 6 after the balloon has been expanded in the tissue wall cavity and the sleeve has torn along its frangible portion in accordance with one non-limiting embodiment of the present invention;

FIG. 9 is a diagram of the surgical instrument of FIG. 6 after the endoscope, balloon and sleeve have been inserted through the tissue wall and the balloon has been deflated in accordance with one non-limiting embodiment of the present invention;

FIG. 10 is a diagram of the surgical instrument of FIG. 6 after the endoscope, balloon and sleeve have been inserted through the tissue wall, the balloon has been deflated and the balloon and sleeve are being retracted into the endoscope lumen in accordance with one non-limiting embodiment of the present invention;

FIG. 11 is a diagram of a surgical instrument including a catheter, a balloon, a frangible sleeve and a needle in accordance with one alternative non-limiting embodiment of the present invention;

FIG. 12 is a diagram of the surgical instrument of FIG. 11 after the balloon has been expanded in the tissue wall cavity and the sleeve has torn along its frangible portion in accordance with one non-limiting embodiment of the present invention;

FIG. 13 is a diagram of the surgical instrument of FIG. 11 after the endoscope, balloon and sleeve have been inserted through the tissue wall and the balloon has been deflated in accordance with one non-limiting embodiment of the present invention;

FIG. 14 is a diagram of the surgical instrument of FIG. 11 after the endoscope, balloon and sleeve have been inserted through the tissue wall, the balloon has been deflated and the balloon and sleeve are being retracted into the endoscope lumen in accordance with one non-limiting embodiment of the present invention;

FIG. 15 is a diagram of the surgical instrument of FIG. 11 as the balloon and sleeve are being retracted into the endoscope lumen in accordance with one non-limiting embodiment of the present invention;

Corresponding reference characters indicate corresponding parts throughout the several views. The exemplifications set out herein illustrate preferred embodiments of the invention, in one form, and such exemplifications are not to be construed as limiting the scope of the invention in any manner.

### DETAILED DESCRIPTION

During the course of various surgical procedures, especially in intraluminal and transluminal procedures, there often exists a need to create a surgical space for advancing surgical instruments or for allowing a surgeon to access a surgical site, for example. Various surgical instruments include expandable balloons to create a surgical space in advance of the surgical instrument. For example, a surgical instrument may include a small needle or guidewire that can be first introduced through an organ wall, for example. A deflated balloon can then be introduced into the hole created by the advancing needle or guidewire. As the balloon is transitioned from a deflated or collapsed position to an inflated or expanded position, the balloon can displace the adjacent tissue, creating a surgical space capable of receiving the advancing surgical instrument.

Among the greatest difficulties in creating a surgical space using a balloon inflation device is maintaining the balloon in an introducible configuration prior to and during introduction of the balloon into the tissue. For example, the deflated balloon can contain pleats that, if not properly and tightly secured, can restrict or complicate advancement of the balloon into the tissue. Further, intraluminal and transluminal procedures often incorporate the use of vacuum-induced lower pressure within delivery trocars or delivery sheaths to encourage advancement of the surgical instrument. For example, an endoscopic trocar may include a source of vacuum pressure to “pull” the endoscopic trocar into the tissue. However, the balloon and any other instrumentation contained within the lumen of the endoscopic trocar can be subjected to the lower pressure within the trocar lumen created by the vacuum. The lower pressure within the trocar lumen, relative to the pressure within the balloon and balloon lumen, can cause the deflated balloon to prematurely expand. Premature expansion of the balloon, even to a minimal degree, greatly complicates advancement of the balloon and the subsequent surgical instrumentation.

These challenges create the need for a sleeve that can be configured to substantially surround a balloon and to maintain the balloon in an introducible configuration during advancement and prior to expansion of the balloon under the direction of the operating surgeon. Further, the sleeve can be configured to include frangible portions that release the sleeve during an operator controlled expansion of the balloon to allow the balloon to dilate the surrounding tissue.

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

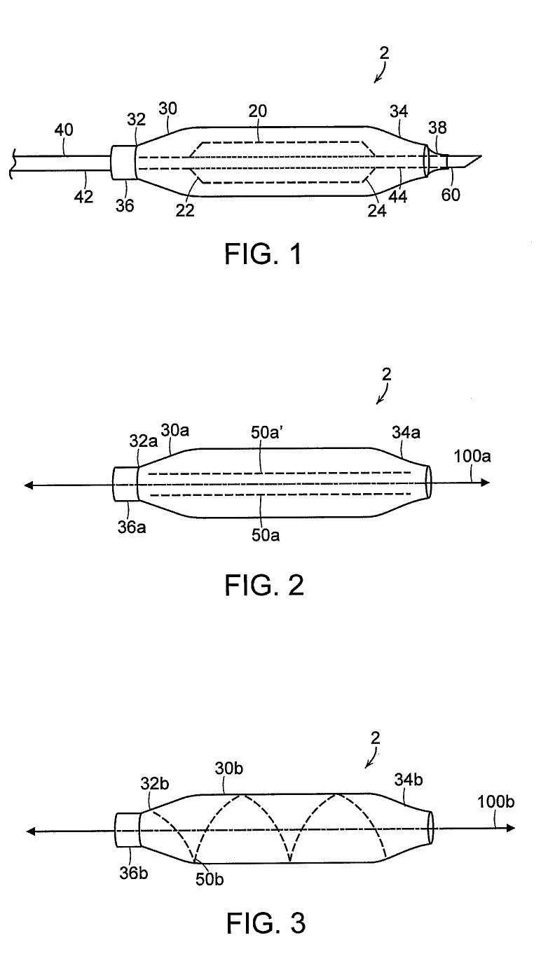

In various embodiments, a surgical instrument and kit can include components which can be configured to manipulate tissue and create a working space within the body of a patient such that a surgeon can easily access, and perform work within, a surgical site using various surgical instruments. In at least one embodiment, referring to FIG. 1, a surgical instrument 2 can include several components, such as catheter 40, balloon 20, sleeve 30, and needle 60, for example, which can be cooperatively utilized in order to easily advance the deflated balloon and to create the working space within the body.

In various embodiments, with continued reference to FIG. 1, balloon 20, which includes balloon proximal end 22 and balloon distal end 24, can be configured to be engaged with catheter 40 at balloon proximal end 22. Catheter 40 can be a balloon catheter in fluid communication with the inflation lumen (not shown) of the balloon, for example, and can be capable of conveying inflation medium to the balloon in order to inflate the balloon under the control of the operating surgeon. In a further embodiment, balloon 20 can be further configured to be engaged with catheter 40 at balloon distal end 24. In various embodiments, catheter 40 can include a catheter proximal portion 42, located proximal to balloon 20 and can be configured to be engaged with balloon 20 at balloon proximal end 22. In a further embodiment, catheter 40 can include a catheter distal portion 44, located distal to balloon 20 and can be configured to be engaged with balloon 20 at balloon distal end 24 and/or with needle 60. The segment of catheter 40 engaged with balloon distal end 24 can be in fluid communication with the inflation lumen of the balloon, however, it is not necessary that this portion of the catheter include a lumen. According to one embodiment, this distal segment of the catheter 40 can be configured to be engaged with needle 60.

In various embodiments, with continued reference to FIG. 1, sleeve 30 can be configured to substantially surround balloon 20 and to be engaged with catheter 40. Sleeve 30 can be configured to surround the entire circumference of balloon 20 or, alternatively, sleeve 30 can be configured to surround a portion of the circumference of the balloon 20 sufficient to retain the balloon 20 in a deflated conformation. Additionally, sleeve 30 can be configured to extend along the entire length of balloon 20 or, alternatively, sleeve 30 can be configured to extend along a portion of the length of the balloon 20 sufficient to retain the balloon 20 in a deflated conformation. Additional variations in the shape and coverage of the sleeve 30 are contemplated such that sleeve 30 can retain the balloon 20 in a substantially deflated conformation prior to operator-induced expansion or inflation of the balloon 20.

According to various embodiments, sleeve 30 can be configured to be engaged with catheter 40 by anchor 36. According to one embodiment, referring to FIG. 1, sleeve 30 can be configured to be engaged with catheter 40 at catheter proximal portion 42 and anchor 36 can be located at sleeve proximal end 32. According to a further embodiment, referring to FIG. 1 1 and described in greater detail below, sleeve 30e can be configured to be engaged with catheter 40 at catheter distal portion 44 and anchor 36e can be located at sleeve distal end 34e. In any event, an anchor can include a suture, a pin, a band, an adhesive, a clip, and/or any other suitable anchor member configured to retain the anchor to the catheter 40. In various embodiments, sleeve 30 and/or anchor 36 can be comprised of any suitable rigid, semi-rigid, or flexible material, such as polycarbonate, PEEK, PTFE, silicone, urethane, or polyolefin, for example.

According to various embodiments, referring to FIG. 1, sleeve 30 can be configured to further include a tapered transition portion 38 located at the sleeve distal end 34. The tapered transition portion 38 can be configured to facilitate the insertion of sleeve 30 into a surgical site and assist in preserving the integrity of and placement of the sleeve 30 when the sleeve 30 is passed through the tissue wall. In at least one embodiment, tapered transition portion 38 can be configured to be engaged with catheter distal portion 44. According to one embodiment, tapered transition portion 38 can be connected to and co-terminus with sleeve 30. According to another embodiment, tapered transition portion 38 can be connected to sleeve 30 through a portion of tapered transition portion 38 containing frangible portions (not shown). According to a further embodiment, tapered transition portion 38 may not be connected to sleeve 30.

In various embodiments, sleeve 30 can include at least one frangible portion 50. As detailed herein, frangible portions can be designed to allow a sleeve to separate, upon expansion of the balloon and tearing of the frangible portions, for example, into one or more than one sleeve portions. Frangible portions can include a perforation including one or more holes, slits or apertures in the sleeve material, for example, or can include the sidewall of the sleeve material surrounding the hole, slit or aperture. Frangible portions also can include a score in the sleeve material, a thin portion of the sleeve material, or a portion of the sleeve material that is adapted to fracture, break, burst or stress upon expansion of the balloon. According to at least one embodiment, each of the resulting sleeve portions can remain anchored to the surgical instrument 2 following the expansion of the balloon in order to facilitate the retrieval and removal of the sleeve portions from the surgical site following the completion of the surgical procedure. In at least one embodiment, referring to FIG. 2, sleeve 30a can be configured to include at least one frangible portion 50a, for example. A frangible portion can include a single hole or aperture formed in the material of the sleeve, as well as a series of holes or apertures formed in any suitable configuration. A frangible portion can be formed in a variety of configurations such as a linear or substantially linear configuration or helical or substantially helical configuration, for example, in the material of the sleeve, to aid in the separation of sleeve 30a into two or more portions 30a, 30′a.

In at least one embodiment, referring to FIG. 2, sleeve 30a can be configured to include a substantially linear arrangement of frangible portions 50a, for example, configured to extend a line substantially parallel to the longitudinal axis 100a of the sleeve 30a. According to one embodiment, frangible portions 50a can extend the entire length of sleeve 30a, from sleeve proximal end 32a to sleeve distal end 34a. According to another embodiment, frangible portions 50a can extend along only a portion of the length of sleeve 30a.

In at least one embodiment, referring to FIG. 3, sleeve 30b can be configured to include a substantially helical arrangement of frangible portions 50b, for example, configured to extend in a substantially helical pattern positioned about the longitudinal axis 100b of the sleeve 30b. According to one embodiment, frangible portion 50b can extend along the entire length of sleeve 30b, from sleeve proximal end 32b to sleeve distal end 34b. According to another embodiment, frangible portion 50b can extend along only a portion of the length of sleeve 30b.

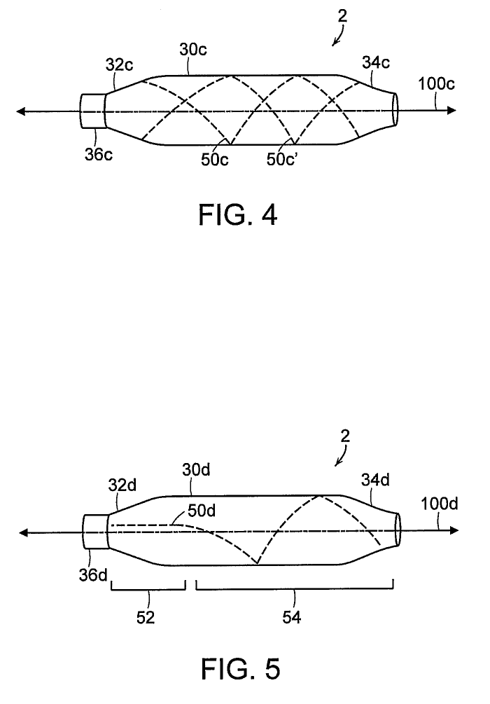

In another embodiment, referring to FIG. 4, sleeve 30c can be configured to include two or more substantially helical frangible portions 50c, 50′c, for example, configured to extend in a helical, or at least substantially helical, pattern positioned about the longitudinal axis 100c of the sleeve 30c. In one embodiment, the two or more helical, or at least substantially helical, frangible portions 50c, 50′c can be congruent, or at least substantially congruent, about the longitudinal axis 100c of the sleeve 30c. In another embodiment, the two or more helical, or at least substantially helical, frangible portions 50c, 50′c may not be substantially congruent. In a further embodiment, the two or more non-congruent substantially helical frangible portions 50c, 50′c can intersect at at least one point of intersection along the length of the sleeve 30c.

In another embodiment, referring to FIG. 5, the sleeve 30d can be configured to include one or more frangible portions 50d, for example, configured to extend in a line parallel, or at least substantially parallel, to the longitudinal axis 100d of the sleeve 30d along a first portion 52 of the sleeve 30d, and to extend in a helical, or at least substantially helical, pattern rotating about the longitudinal axis 100d of the sleeve 30d along a second portion 54 of the sleeve 30d. In a further embodiment, not depicted, the sleeve 30d can be configured to include two or more portions 52 and 54, for example. Each such portion can be co-terminus with an adjacent portion(s) and have a different configuration of frangible portions than the adjacent portion(s).

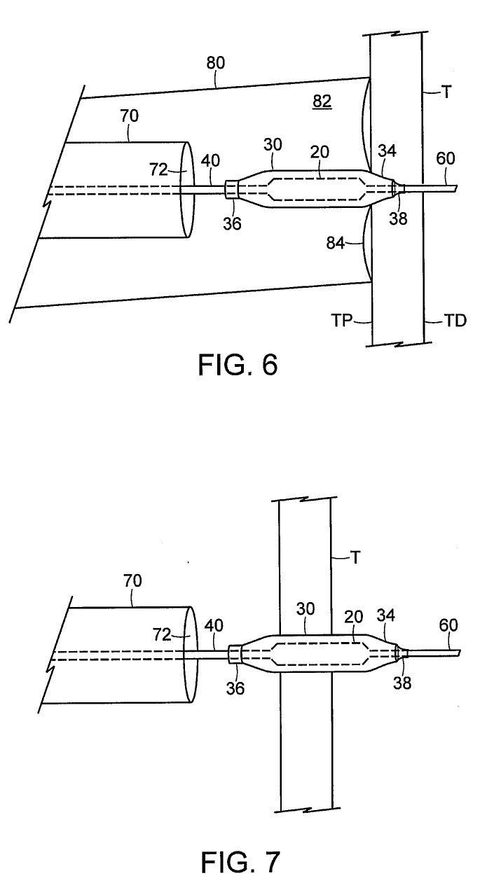

In at least one embodiment, referring to FIG. 6, the surgical instrument 2 of FIG. 1 can be configured to further include an endoscope 70. The endoscope 70 can be cylindrical, or at least substantially cylindrical, and define an endoscope lumen 72 extending along the length of the endoscope 70. In at least one embodiment, the catheter 40, balloon 20 and/or sleeve 30 can be configured to be slidably received within the endoscope lumen 72.

In another embodiment, the surgical instrument 2 can be configured to further include a delivery sheath (not shown). The delivery sheath can be cylindrical, or at least substantially cylindrical, and define a sheath lumen (not shown) extending along the length of the delivery sheath. In at least one embodiment, the catheter 40, balloon 20 and/or sleeve 30 can be configured to be slidably received within the delivery sheath.

In at least one embodiment, the surgical instrument 2 can be configured to further include a trocar 80. The trocar 80 can be cylindrical, or at least substantially cylindrical, and define a trocar lumen 82 extending substantially along the length of the trocar 80. In at least one embodiment, the endoscope 70 can be configured to be slidably received within the trocar lumen 82. In a further embodiment, the catheter 40, balloon 20 and/or sleeve 30 can be configured to be slidably received within the trocar lumen 82.

In various embodiments, the surgical instrument 2 can be inserted into a patient's body. In at least one embodiment, referring to FIG. 6, trocar 80 can be inserted into the patient's body and advanced to a tissue wall “T”. According to one embodiment, endoscope 70 can be slidably received within the trocar lumen 82 of trocar 80 and positioned proximal to the distal end 84 of trocar 80. In a further embodiment, catheter 40, balloon 20, sleeve 30 and/or needle 60 can be slidably received within the endoscope lumen 72. In various embodiments, the needle 60 can be advanced through the tissue wall “T”, from the proximal tissue wall surface “TP” through to the distal tissue wall surface “TD”. Following advancement of the needle 60, the balloon 20 and/or surrounding sleeve 30 can be advanced through the tissue wall “T”.

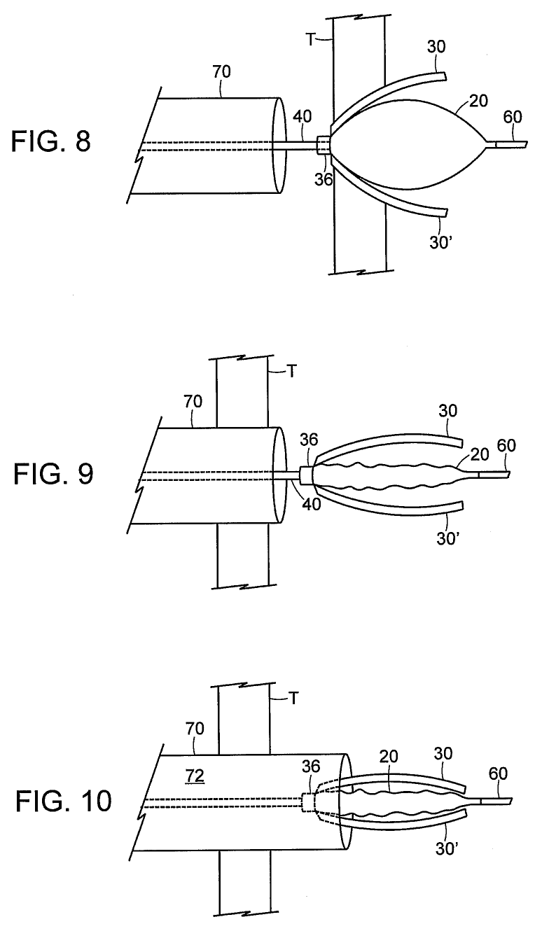

In various embodiments, referring to FIG. 7, the balloon 20 and/or surrounding sleeve 30 can be advanced through the tissue wall “T” until the balloon is substantially in position for expansion. Referring to FIG. 8, in one embodiment, the balloon 20 can be expanded or inflated such that it contacts the tissue wall “T” and expands the opening or cavity within the tissue wall “T”, which was created by needle 60, for example. In various embodiments, upon expansion or inflation of the balloon 20, the sleeve 30 can tear along the one or more frangible portions 50, 50′, resulting in the creation of one or more sleeve portions 30, 30′. According to the embodiment illustrated in FIG. 8, the sleeve 30 can be configured to be engaged with the catheter 40 at the catheter proximal portion 42 by anchor 36, located at the sleeve proximal end 32. Upon expansion of the balloon 20 and creation of the one or more sleeve portions 30, 30′, the sleeve portions 30, 30′ can maintain engagement with the anchor 36 at the sleeve proximal end 32.

In various embodiments, referring to FIG. 9, following expansion of the balloon 20, endoscope 70 can be advanced into the dilated opening or cavity within the tissue wall “T”. In one embodiment, following the advancement of the endoscope 70 or the trocar 80 through the dilated opening or cavity within the tissue wall “T”, the balloon 20 can be deflated to allow retraction of the balloon 20 in a proximal direction into the endoscope 70 or the trocar 80. As illustrated in FIG. 10, in one embodiment, the deflated balloon 20 can be retracted proximally into the endoscope lumen 72 or the trocar lumen 82. According to one embodiment, the deflated balloon 20 and sleeve portions 30, 30′ can be retracted proximally into the endoscope lumen 72 and removed from the patient's body. According to another embodiment, the deflated balloon 20 and needle 60 can be further advanced distally into the tissue wall “T” or into a subsequent tissue wall and the expansion and tissue dilation procedure can be repeated to further advance the endoscope 70 or trocar 80 into the patient's body.

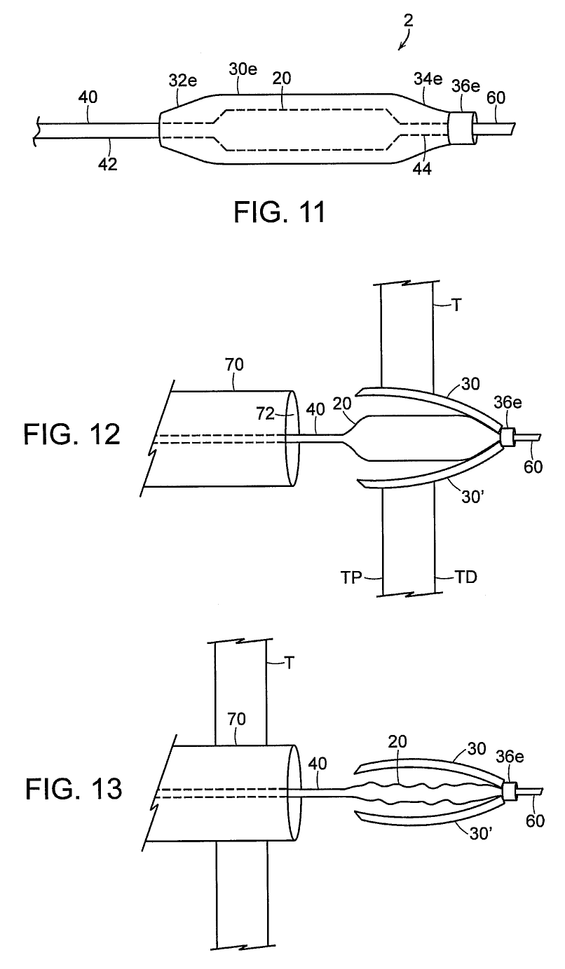

In various alternative embodiments, referring to FIG. 11, anchor 36e can be configured to be engaged with the sleeve 30e at sleeve distal end 34e. In one embodiment, anchor 36e can be configured to be engaged with catheter 40 at catheter distal portion 44. In another embodiment, anchor 36e can be configured to be engaged with balloon 20 at balloon distal end 24. In various embodiments, anchor 36e can be configured to form a tapered transition portion, similar to tapered transition portion 38, described above, to assist in preserving the integrity and placement of the sleeve when it is passed through the tissue wall.

In various embodiments, referring to FIG. 12, the surgical instrument 2 can be inserted into a patient's body. In at least one embodiment, similar to the above, trocar 80 (not shown) or endoscope 70 can be inserted into the patient's body and advanced to a tissue wall “T”. According to one embodiment, catheter 40, balloon 20, sleeve 30 and/or needle 60 can be slidably received within the endoscope lumen 72. In various embodiments, the needle 60 can be advanced through the tissue wall “T”, from the proximal tissue wall surface “TP” through to the distal tissue wall surface “TD”. Following advancement of the needle 60, the balloon 20 and/or surrounding sleeve 30 can be advanced through the tissue wall “T”.

In various embodiments, referring again to FIG. 12, the balloon 20 and surrounding sleeve 30 can be advanced through the tissue wall “T” until the balloon is substantially in position for expansion. Referring to FIG. 12, in one embodiment, the balloon 20 can be expanded or inflated to contact the tissue wall “T” to expand or dilate the opening or cavity within the tissue wall “T”. In various embodiments, upon expansion or inflation of the balloon 20, the sleeve 30 can tear along the one or more frangible portions 50, 50′, resulting in the creation of one or more sleeve portions 30, 30′. According to the embodiment illustrated in FIG. 12, the sleeve 30 can be configured to be engaged with the catheter 40 at the catheter distal portion 44 by anchor 36, located at the sleeve distal end 34. Upon expansion of the balloon 20 and creation of the one or more sleeve portions 30, 30′, the sleeve portions 30, 30′ can maintain engagement with the anchor 36 at the sleeve distal end 34.

In various embodiments, referring to FIG. 13, following the expansion of the balloon 20, endoscope 70 can be advanced into the dilated opening or cavity within the tissue wall “T”. In one embodiment, following the advancement of the endoscope 70 or the trocar 80 through the dilated opening or cavity within the tissue wall “T”, the balloon 20 can be deflated to allow the retraction of the balloon 20 into the endoscope 70 or the trocar 80, as illustrated in FIG. 13.

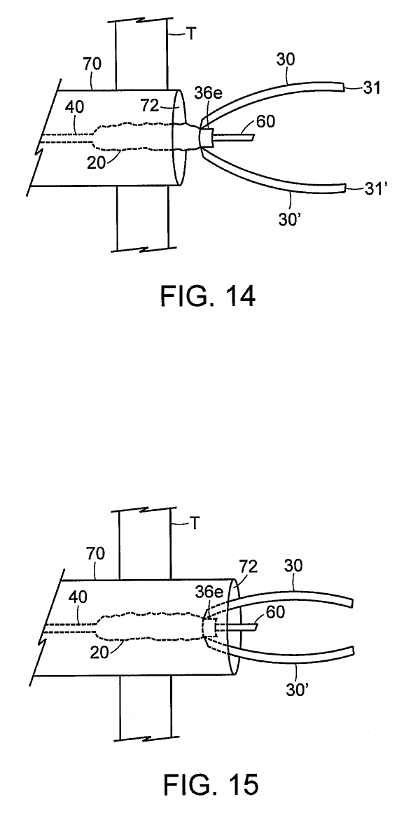

As illustrated in FIG. 14, in one embodiment, the deflated balloon 20 can be retracted proximally into the endoscope lumen 72 or the trocar lumen 82. In various embodiments, the sleeve portions 30, 30′ can be configured to be engaged with the anchor 36 at the sleeve distal end 34. Accordingly, upon proximal retraction of the balloon 20, the sleeve portions 30, 30′ can be restrained by the frictional force of the adjacent tissue such that the sleeve portions 30, 30′ remain stationary and eventually invert their orientation such that the unsecured ends 31, 31′ of the sleeve portions 30, 30′ extend distally beyond the sleeve distal end 34 and distally beyond the anchor 36.

According to one embodiment, referring to FIG. 15, the deflated balloon 20 and sleeve portions 30, 30′ can be retracted proximally into the endoscope lumen 72 and removed from the patient's body. According to another embodiment, the deflated balloon 20 and needle 60 can be further advanced distally into the tissue wall “T” or into a subsequent tissue wall and the expansion procedure is repeated to further advance the endoscope 70 or trocar 80 into the patient's body.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument can be obtained and if necessary cleaned. The instrument can then be sterilized. In one sterilization technique, the instrument can be placed in a closed and sealed container, such as a plastic or TYVEK bag. The container and instrument can then be placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

While this invention has been described as having exemplary designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A surgical instrument for use in manipulating tissue within the body of a patient, the instrument comprising:
a balloon, wherein said balloon is configured to be transitioned from a collapsed position to an expanded position; and
a sleeve, wherein said sleeve includes at least one frangible portion and wherein said sleeve is configured to at least partially surround said balloon when said balloon is in the collapsed position and to at least partially separate along said at least one frangible portion when said balloon is in the expanded position.

2. The surgical instrument of claim 1, wherein said sleeve further comprises a longitudinal axis and wherein said frangible portion is configured to extend in at least one line substantially parallel to said longitudinal axis of said sleeve.

3. The surgical instrument of claim 2, wherein said frangible portion is configured to extend in two, three, or four lines, wherein each line is substantially parallel to said longitudinal axis of the sleeve.

4. The surgical instrument of claim 1, wherein said sleeve further comprises a longitudinal axis and wherein said frangible portion is configured to extend in at least one substantially helical pattern positioned about said longitudinal axis of said sleeve.

5. The surgical instrument of claim 1, wherein said sleeve further comprises a longitudinal axis and wherein said frangible portion is configured to extend along at least two substantially congruent helical patterns positioned about said longitudinal axis of said sleeve.

6. The surgical instrument of claim 5, wherein said at least two substantially congruent helical patterns do not intersect along a central portion of said sleeve.

7. The surgical instrument of claim 1, wherein said sleeve further comprises a longitudinal axis and wherein said frangible portion is configured to extend along a pattern, wherein said pattern is substantially parallel to said longitudinal axis of said sleeve along a first portion of said sleeve and said pattern is substantially helically positioned about said longitudinal axis of said sleeve along a second portion of said sleeve.

8. The surgical instrument of claim 1, wherein said frangible portion comprises a perforation.

9. The surgical instrument of claim 1, wherein said frangible portion comprises a score.

10. The surgical instrument of claim 1, wherein said instrument further comprises a catheter configured to be attached to a proximal portion of said balloon and, optionally, further configured to be attached to a distal portion of said balloon.

11. The surgical instrument of claim 10, wherein said sleeve further comprises an anchor portion at at least one end of said sleeve.

12. The surgical instrument of claim 11, wherein said anchor portion of said sleeve is at a proximal portion of said sleeve and is configured to be attached to a portion of said catheter adjacent to a proximal portion of said balloon.

13. The surgical instrument of claim 12, wherein said sleeve further comprises a tapered transition portion at a distal portion of said sleeve configured to be located adjacent to a distal portion of said balloon.

14. The surgical instrument of claim 11, wherein said anchor portion of said sleeve is at a distal portion of said sleeve and is configured to be attached to a portion of said catheter adjacent to a distal portion of said balloon.

15. The surgical instrument of claim 1, wherein said instrument further comprises an endoscope including an endoscope lumen extending along a longitudinal axis of said endoscope, and wherein said balloon and said sleeve are slidably received within said endoscope lumen.

16. The surgical instrument of claim 15, wherein said instrument further comprises a trocar including a trocar lumen extending along a longitudinal axis of the trocar, and wherein said endoscope is slidably received within said trocar lumen.

17. A surgical kit for use in manipulating tissue within the body of a patient, the kit comprising:
a balloon, wherein said balloon is configured to be transitioned from a collapsed position to an expanded position;
a sleeve, wherein said sleeve includes at least one frangible portion and wherein said sleeve is configured to at least partially surround said balloon when said balloon is in the collapsed position and to at least partially separate along said at least one frangible portion when said balloon is in the expanded position; and
a catheter, wherein said catheter is configured to be attached to at least a portion of said balloon.

18. The surgical kit of claim 17, further comprising an endoscope, wherein said endoscope further comprises an endoscope lumen and wherein said catheter, said balloon and said sleeve are slidably received within said endoscope lumen.

19. The surgical kit of claim 18, further comprising a trocar wherein said trocar includes a trocar lumen extending along a longitudinal axis of said trocar, and wherein said endoscope is slidably received within said trocar lumen.

20. A surgical instrumentation system comprising:
an inflation means, wherein said inflation means is configured to be transitioned from a collapsed position to an expanded position; and
a covering means, wherein said covering means includes a separation means and wherein said covering means is configured to at least partially surround said inflation means when said inflation means is in the collapsed position and to at least partially separate along said separation means when said inflation means is in the expanded position.

21. The surgical instrumentation system of claim 20, wherein said system further comprising a delivery means including a delivery lumen and wherein said inflation means and said covering means are slidably received within said delivery lumen.
